(19) United States
(12) Patent Application Publication
(10) Pub. No.: US 2008/0009023 A1
(43) Pub. Date: Jan. 10, 2008

(54) Diagnostic Method For The Forecasting Of Transplanted Organ Loosing

(76) Inventors: Paola Romagnani, Firenze (IT); Mario Rotondi, Torre Del Greco (IT); Mario Serio, Bagno A Ripoli (IT)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 11/791,350

(22) PCT Filed: Nov. 24, 2005
(86) PCT No.: PCT/EP05/56186

§ 371 Date: May 22, 2007

(30) Foreign Application Priority Data
Nov. 25, 2004 (IT) FI2004A000243

(51) Int. Cl. G01N 33/53; G01N 33/68

(52) U.S. Cl. 435/7.92; 436/86

(57) ABSTRACT

It is described a diagnostic method which allows to establish the risks of rejection of a transplanted organ by measuring the pre-transplant concentration of Mig in the serum of a patient to be submitted to transplantation.

### FIELD OF THE INVENTION

The invention is related to serum markers for diagnostic use.

### STATE OF ART

The search of new serum markers able to predict the immune response of a subject programmed to receive an organ transplantation is an important target of clinical interest.

The sole parameter used till now in the attempt to modulate the immunosuppressive therapy on the patient is the panel of reactive antibodies (PRA).

It is well known (Am. J. Transplant. 4, 1466-1474, 2204) that the increased pre-transplantation serum levels of chemokine IP-10/CXCL10, which plays an important role in the pathogenesis of acute and chronic organ rejection, are strictly related to the risk of allograph loss.

The CXCL10, together with the other CXCR3 chemokine receptor ligands, CXCL9 (Mig) and CXCL11 (I-TAC), plays a double biological role in the allograph rejection.

These chemokines induce the migration of lymphocytes, dentritic cells, macrophages and other immune cells and modulate the angiogenesis which is an other important component of inflammation.

The described biological functions of the above mentioned chemokines are the basis of organ rejection,

It is evident the importance of availability of serum markers able to give us a prevision of the immunological reactivity of a subject who undergoes organ transplantation, and if possible with greater sensitivity, than with the previously utilized markers.

### DETAILED DESCRIPTION OF THE INVENTION

We discovered that elevated serum levels of the chemokine CXCL9 provoke an extremely high risk of organ rejection. The risk is statistically higher than that of elevated serum levels of IP-10. Therefore CXCL9 represents the best serum marker for prevision of organ transplantation rejection.

We would like to point out that the serum levels of the other “sister” chemokine CXCL11 are unable to give any prevision of transplantation rejection.

The reagents and the methods used for the present invention are well known, for example Enzyme Linked-Immuno-Sorbent Assay (ELISA) using anti-CXCL9 antibodies.

Experimental Part

Serum levels before transplantation of CXCL9 and CXCL11 have been measured in 213 subjects who had a follow-up of 5 years and in 17 healthy controls. In the same subjects CXCL10 serum levels were also measured for comparison. In controls the serum levels of CXCL9 were 52.4±15.3 pg/ml while in the transplanted subjects the pre-transplantation serum levels were 255.6±14.99 pg/ml. The CXCL9 levels were higher in transplanted patients who have lost the allografh comparison to those who have kept the organ (453.27±61.5 versus 241.7±15.01). The CXCL11 resulted unusable for organ rejection prevision because the above mentioned chemokine was undetectable in the majority of patients.

The survival curves according Kaplan-Meier were performed in 300 subjects divided in 4 groups on the basis of serum levels of CXCL9 (0-25 centile=<121.4 pg/ml, n=53), (25-50 centile=between 121.4 and 194.3 pg/ml, n=64), (50-75 centile=between 194.3 and 312.3 pg/ml, n=53), (75-100 centile=>312.3 pg/ml, n=53). These curves showed a progressive reduction of survival at 5 years distance of transplanted organ proportional to the pre-transplantation levels of CXCL9, 98.1% for group 1, 100% for group 2, 96.2% for group 3 and 79.2% for group 4 (p<0.001 in total; p<0.001 group 1 versus group 4; p<0.001 group 2 versus group 4 and p<0.001 group 3 versus group 4).

To establish the relative risks of organ decreased function in the subjects with elevated levels of CXCL9 before transplantation (>312.3 pg/ml) a multivariated analysis according Cox considering allografh loss as depending variable has been performed. Age, sex of recipient, number of mismatches HLA-A, HLA-B and HLA-DR, the primitive disease, the type of immunosuppression, the number of transplantations, the time of cold ischemia, the number of rejections, age and sex of donor have been considered as co-variables in the analysis.

The result showed that serum levels of CXCL9 above 312.3 pg/ml induce a significant increase of rejection risk (risk ratio 10.390; C.I. 1.646-65.575; p=0.013) in the patients.

Another co-variated analysis was performed including CXCL10 as co-variable. Once again the pre-transplantation CXCL9 serum levels above 312.3 pg/ml determined an increased risk of organ loosing in the patients (risk ratio 10.433; CJ. 1.597-68.146; p=0.01). The other co-variables did not give a statistical significance with exception of pre-transplantation serum levels of CXCL10 which, when considered in absence of CXCL9, gave an increased risk of organ loosing but significantly lower than that obtained by CXCL9. On the basis of the above reported results the pre-transplantation serum levels of CXCL9 are able to select with highest risk of developing acute and/or chromo organ rejection and consequently with the highest risk of organ loosing. These patients should be treated with more potent immunosuppressive therapies to avoid organ loss.

The diagnostic method according to invention is useful to predict acute and/or chronic rejection and consequently organ loosing in kidney, heart, liver, bone marrow, lung, pancreas or pancreatic islets and intestine transplantation.

What is claimed is:

1. Diagnostic method for prevision of transplanted organ loosing by measurement of pre-transplantation serum levels of CXCL9 (MIG).

2. Method according claim 1 for prevision of acute rejection of transplanted organ.

3. Method according to claim 1 for prevision of chronic rejection of transplanted organ.

4. Method according to claims 3 in which the transplanted organ is kidney, heart, liver, bone marrow, lung, pancreas or pancreatic islets, intestine.

5. Method according to claims 1 in which the serum level is measured by Enzyme Linked-Immuno-Sorbent Assay (ELISA).
